(19) United States
(12) Patent Application Publication
(10) Pub. No.: US 2006/0241379 A1
(43) Pub. Date: Oct. 26, 2006

(54) Method for imaging of a periodically-moving subject region of a subject

(76) Inventors: Andreas Greiser, Uttenreuth (DE); Wilfried Landschutz, Baiersdorf (DE); Peter Speier, Erlangen (DE)

Correspondence Address: SCHIFF HARDIN, LLP;PATENT DEPARTMENT, 6600 SEARS TOWER, CHICAGO, IL, 60606-6473, US

(21) Appl. No.: 11/324,376

(22) Filed: Jan. 3, 2006

(30) Foreign Application Priority Data
Jan. 3, 2005 (DE) 10 2005 000 714.7

(51) Int. Cl. A61B 5/05

(52) U.S. Cl. 600/410

(57) ABSTRACT

In a method for imaging a periodically-moving subject region of a subject, an overview image data set is initially obtained that maps a movement of the subject region, at least two positions that the subject region assumes at corresponding points in time are marked in the overview image, further positions of the subject region at further points in time are interpolated from the marked positions and further points in time, and a subsequent diagnostic imaging of the moving subject region is implemented using the marked and interpolated positions.

### BACKGROUND OF THE INVENTION

1. Field of the Invention

The present invention concerns a method for imaging a periodically-moving subject region of a subject, in particular a method for medical imaging of moving organs.

2. Description of the Prior Art

The demands on imaging methods for moving organs are high. In order to distinctly map moving regions, in addition to a good spatial resolution the imaging method must also possess a sufficient temporal resolution. If slower imaging methods are used for specific reasons such as, for example, a high spatial resolution, the acquisition region for the imaging can be guided along corresponding to the movement. The use of correction methods in the post-processing of the acquired image data is also known.

Sequences that can acquire the movement of an imaging slice with sufficiently-high temporal resolution are used today for dynamic magnetic resonance imaging of moving organs such as, for example, the heart. The imaging sequences used for this purpose can be divided into real-time methods, in which the entire slice is usually acquired over the movement such that the movement itself is shown with sufficient temporal resolution, and segmented methods, known in which only a part of the total data required for the imaging of the slice is acquired from each movement state for a movement cycle. By multiple repetition of the image acquisition over multiple movement cycles, the entire image information ultimately can be acquired. Since the achievable spatial resolution is currently very limited in the real-time method, in many applications the only way to achieve a diagnostic image quality is to use segmented acquisition methods.

Methods for tracking the image acquisition region have been established in magnetic resonance imaging. Techniques such as, for example, the navigator technique or the PACE (prospective acquisition correction) technique enable slice tracking given a moving subject on the basis of additional position information acquired in real time. These methods, however, normally enable only slice tracking perpendicular to a fixed slice orientation. Moreover, the correlation between the measured position information and the actual slice to be shown must be known. The momentary movement state or the position is then determined with a sharp contrast change at the displacement of areas. A disadvantage of these techniques is that a portion of the acquisition time must be devoted to the acquisition for the navigator signal.

If the physiological movement has a periodic curve, which is the case to a good approximation for cardiac contraction, a priori information can be used for optimization of the actual measurement for imaging. The use of the navigator techniques then can be limited in part. From U.S. Pat. No. 6,792,066 (corresponding to DE 102 21 642 A1) it is known to determine the movement cycle in a cine scan in advance of the actual measurement. Displacements and/or tipping of the slice to be imaged are thereby determined in a slice orientation essentially perpendicular to the slice orientation required later. A sequence of time-dependent slice position markings is initially set in the reference images, with a time markers respectively being associated with the individual slice position markings. The positions of the subsequent slice images to be acquired are then determined by means of this sequence of time-dependent slice position markings, dependent on an acquisition point in time of the respective slice image relative to a reference point in time.

A technique in which the movement cycle of the marked plane can be tracked over (throughout) the heart movement by display of a tagging pattern (for example a line) at the beginning of a heart cycle with subsequent cine imaging is known from the article by Kozerke, Scheidegger, Pedersen, Boesiger: “Heart Motion Adapted Cine Phase-Contrast Flow Measurements Through the Aortic Valve”, appearing in 1999 in Magnetic Resonance in Medicine, volume 42, pages 970 through 978. The suitable slice geometries (thus the position and orientation of the slice to be acquired) for all heart phases can be extracted with a dynamic image analysis, but limitations exist with regard to the positioning of the tagging pattern. In the cited article, the actual slice to be shown is thus not marked by a tagging line, but rather by a slice displaced relative thereto. An alternative, manual positioning of all (typically 20 to 30) slices for the primary measurement is not acceptable from the viewpoint of the operator and workflow because this takes too long the large number of slices can be placed accurately relative to one another only with difficulty.

### SUMMARY OF THE INVENTION

An object of the present invention is to provide a method for imaging a periodically-moving subject region that is simple to administer and proceeds quickly and in a robust manner.

This object is achieved in accordance with the invention by initially obtaining an overview image data set that images the movement of the subject region, at least two positions that the subject region assumes at at least two various points in time are subsequently marked on the overview image data set, further positions of the subject region at further points in time are subsequently interpolated from the marked positions and the corresponding points in time; and a subsequent imaging of the moving subject region is implemented by use of the marked and interpolated positions.

In comparison to a fixed slice positioning in the imaging of the moving subject region, a higher diagnostic value of the image data results because the subject region is imaged in its full movement. Relative to a manual positioning of all individual slices, a significant time savings results for the user. Since no navigator techniques are used in the present method, the image generation is shorter overall. Moreover, it is not necessary to evaluate a contrast change in order to determine the current movement state. The placement of the slice can ensue directly on the desired anatomy. A tagging-based reference scan can also be used as an overview image data set that can be combined for visual orientation in the event that the positioning of the slices at various points in time is possible in an easy manner on the basis of a shifting tagging pattern.

The geometries of all points in time can be determined over the movement cycle with the marking of two positions that the moving subject region assumes at two different points in time and that, for example, represent the extreme positions of the slice to be represented and with the association of the corresponding points in time at which these positions are achieved by the slice to be represented. Both time-position pairs (or, if the subject region is limited to an acquisition slice, also time-slice geometry pairs) are used in order to scale an interpolation function in the space and time direction. As used herein “slice geometry” means the position and the orientation of the slice to be mapped. This principle can be applied to all parameters characterizing the slice geometry such as displacement, tilting or position of the normal vector and rotation in the slice place (in-plane rotation).

When a larger number of slices are established with the associated points in time, pathologies that exhibit a characteristic deviation of the movement cycle from the norm can be imaged. A compromise between high precision with many sampling points and a minimum time expenditure for the measurement preparation with few sampling points then ensues specific to the examination.

### DESCRIPTION OF THE DRAWINGS

FIG. 1 schematically illustrates of a diagnostic magnetic resonance apparatus for imaging of a periodically-moving subject region of a subject, operable in accordance with the inventive method.

FIG. 2 is a flowchart of the basic method steps for imaging of a periodically-moving subject region, in accordance with the invention.

FIG. 3, in an overview representation, shows the position of a first slice plane at a first point in time.

FIG. 4 shows the position of a second slice plane for imaging of the same subject region as in FIG. 3 at a second point in time.

FIG. 5 shows an interpolation function for calculation of the slice geometry of further slice planes.

### DESCRIPTION OF THE PREFERRED EMBODIMENTS

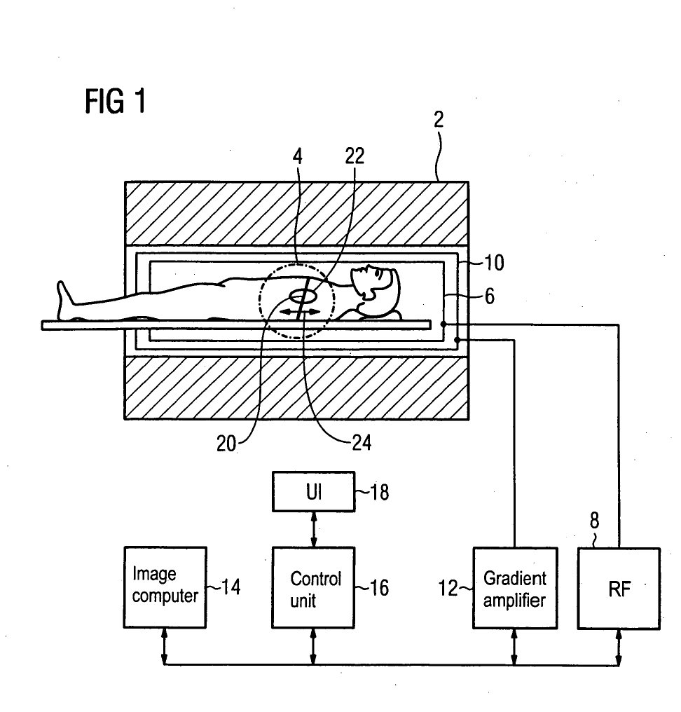

FIG. 1 shows the design of a diagnostic magnetic resonance apparatus with which imaging of a moving subject region of a moving subject can be implemented. The magnetic resonance apparatus has a conventional design except its controller is designed for execution of an embodiment of the inventive method.

Since the basic design of a diagnostic magnetic resonance apparatus as well known, here only the basic functional components are mentioned in brief summary. The magnetic resonance apparatus comprises a superconducting magnet 2 that generates a constant and homogeneous magnetic field in an imaging region 4 in its cylindrical inner chamber. A radio-frequency antenna unit 6 for excitation and reception of magnetic resonance signals is located in the cylindrical inner chamber. The radio-frequency antenna unit 6 is connected with a radio-frequency transmission and reception unit 8. A gradient coil unit 10 for spatially coding the magnetic resonance signals with temporally- and spatially-variable magnetic gradient fields is likewise arranged in the inner chamber of the magnet 2. The currents required for this purpose are supplied form a gradient amplifier unit 12. An image computer 14 reconstructs corresponding slice images from the received and spatially-coded magnetic resonance signals. A controller 16 (realized by a computer with a control program) controls the entire measurement workflow and the image generation. A user interface (U1) 18 is connected with the controller 16, the user interface 18 in general including a monitor, an input keyboard and a mouse or another operating element for a cursor on the monitor.

For planning a magnetic resonance examination (data acquisition) for a diagnostically-meaningful imaging, overview images often are initially generated that are used to determine the position and alignment of the slice planes for the diagnostic imaging, by means of graphical slice positioning. When a specific slice 20 is to be mapped within a moving organ 22 (such as, for example, the heart), a different position of the slice plane results at each point in time. The periodic displacement of the slice to be imaged (the subject region to be imaged) is symbolized in FIG. 1 by a double arrow 24.

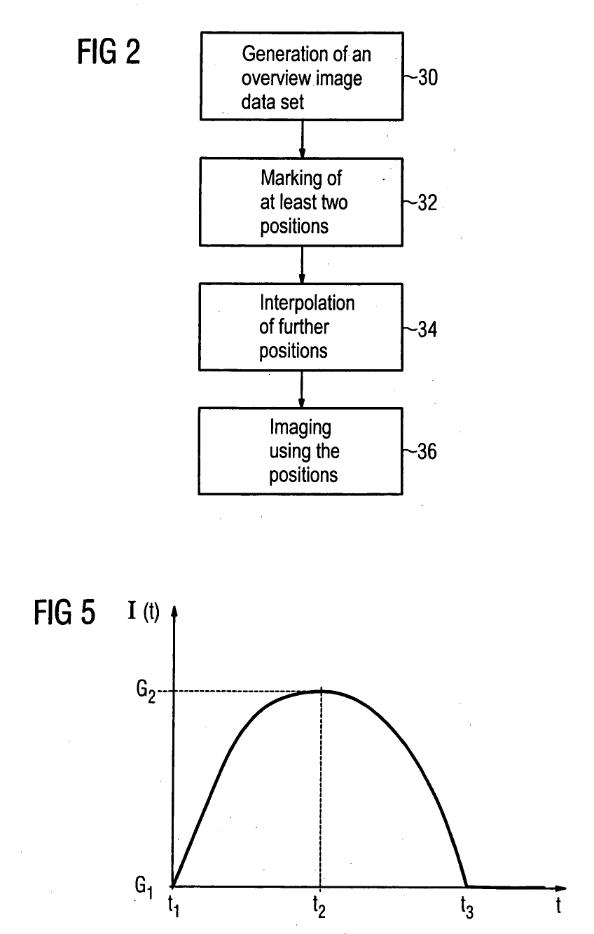

FIG. 2 shows the basic method steps for imaging a moving subject region of a subject in accordance with the invention. In a first step 30, an overview image data set is generated with a suitable fast magnetic resonance sequence such as, for example, a cine TrueFISP sequence, in a slice plane in which the movement of the subject region to be imaged can be represented and analyzed well. The movement of the subject region is thereby completely acquired at sufficient temporal resolution with individual overview images.

The extreme positions, for example, in the movement of the subject region are marked in a subsequent marking step 32. The movement from the first extreme position to the second extreme position can, for example, be described by a displacement, a tilting and a rotation of the second extreme position established in the plane relative to the first extreme position.

In an interpolation step 34, further positions at further points in time are determined from the marked positions and the corresponding points in time by means of a suitable interpolation function.

Finally, imaging 36 of the moving subject region using the previously marked and also interpolated positions ensues with an imaging sequence that supplies a spatial and temporal resolution of the image data sufficient for a diagnosis. For example, a cine TrueFISP or FLASH contrast is used for heart imaging.

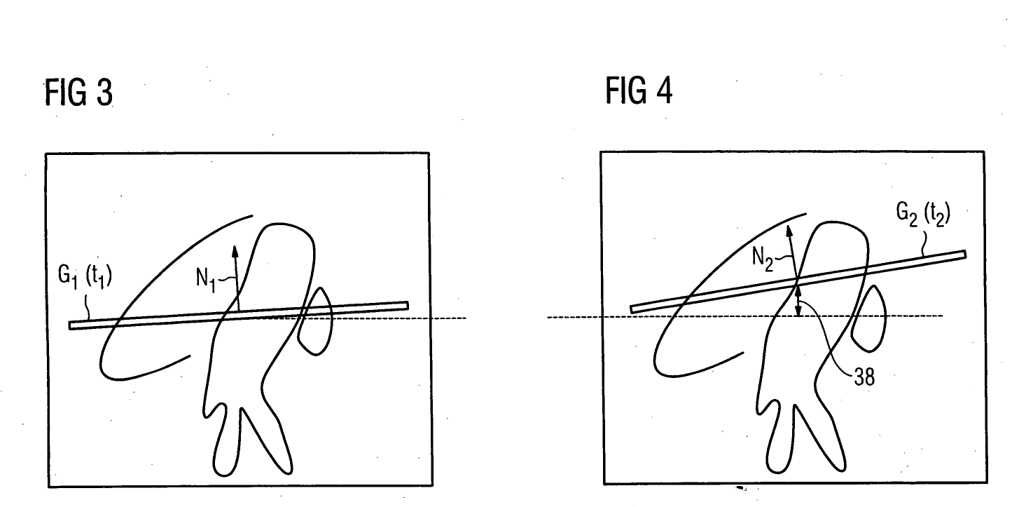

The marking of two positions that the subject region assumes in the course of the movement is explained using FIG. 3 and FIG. 4. FIG. 3 shows the position of a region to be imaged of a moving subject in a first extreme position that is determined by the slice geometry G₁(t₁). The normal vector N₁ of this slice geometry G₁(t₁) is still shown. The normal vector generally serves for the determination of a tilting or rotation of the region to be mapped. FIG. 4 shows a second extreme position of the same region to be mapped, defined by a slice geometry G₂(t₂). Here as well the normal vector N₂ of this slice geometry is still plotted in the image. It can be seen that the slice position G₂(t₂) can be transformed from the slice position G₁(t₁) by a displacement 38 and a tilting (see the changed alignment of both normal vectors N₁ and N₂). A rotation in the slice plane also can be used, as needed.

For intervening points in time, further slice geometries are interpolated between the extreme positions of the subject region or both extreme slice geometries and the associated points in time at these positions are estimated. The interpolation function is determined by a typical movement of the moving subject region. FIG. 5 shows an interpolation function that is suitable for the slice displacement of the heart valve plane of the heart. The interpolation function is defined per segment and rises sinusoidally from the point in time t₁ until the point in time t₃ and subsequently falls (again sinusoidally) from the region t₂ to t₃. For points in time after t₃, the interpolation function is zero. The value zero of the interpolation function means that the slice geometry at the point in time t₁ is completely determined by the first slice geometry defined in the image series. Exactly the second slice geometry is used at the maximum of the interpolation function. For all other points in time between both points in time of the extreme positions, the corresponding slice geometries are determined from the amplitude of the interpolation function and the geometries of both geometry positions. This correlation can be described according to a formula as follows:
Gᵢ(t)=I(t)G₁+(1−I(t))G₂,
wherein Gᵢ(t) describes the interpolated slice geometries at the point in time t and I(t) describes the interpolation function already mentioned above.

The preceding method explained using two slice geometries can be applied for a larger number of marked slice geometries with the associated points in time in order to enable the individual adaptation of the interpolation function to the dynamic information predetermined by the overview image data set. This is particularly advantageous given pathologies since characteristic deviations of the movement workflow from the norm must then be taken into account.

The preceding method has been using magnetic resonance imaging as an example, but it can also be used in other imaging modalities, such as, for example, ultrasound imaging.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for imaging a periodically moving region of an examination subject, comprising the steps of:
generating an overview image data set that images movement of a periodically moving region of a subject;
in said overview image data set, electronically marking at least two positions that the periodically moving region assumes at respective points in time;
automatically electronically interpolating further positions of said region at further points in time from said marked positions and said further points in time; and
obtaining a diagnostic image of said periodically moving region of said subject using said marked and interpolated positions.

2. A method as claimed in claim 1 wherein the step of electronically marking at least two positions in said overview image data set comprises electronically marking at least one extreme position of said periodically moving region in said overview image data set.

3. A method as claimed in claim 1 wherein the step of electronically marking at least two positions in said overview image data set comprises electronically marking two extreme positions of said periodically moving region in said overview image data set.

4. A method as claimed in claim 1 comprising determining said marked positions and said interpolated positions from a displacement of said region due to the periodic movement.

5. A method as claimed in claim 1 wherein said region in said overview image data set exhibits a normal vector, and comprising determining said marked positions and said interpolated positions from a change of said normal vector.

6. A method as claimed in claim 1 comprising determining said marked positions and said interpolated positions by rotation of said region of said subject.

7. A method as claimed in claim 1 wherein the step of automatically electronically interpolating said further positions comprises automatically electronically applying an interpolation function to said overview image data set.

8. A method as claimed in claim 7 comprising employing an interpolation function that is defined in segments.

9. A method as claimed in claim 7 comprising employing an interpolation function that is comprised of a plurality of sinusoidal segments.

10. A method as claimed in claim 9 comprising employing an interpolation function exhibiting a sinusoidal curve between extreme positions of the periodic movement of the region.

11. A method as claimed in claim 1 comprising obtaining said overview image data set by magnetic resonance imaging, and obtaining said diagnostic image by magnetic resonance imaging.

12. A method as claimed in claim 1 wherein the step of obtaining an overview image data set comprises obtaining an overview image data set in a slice plane of the heart of subject mapped in a cine image.
